(19) United States
(12) Patent Application Publication
(10) Pub. No.: US 2021/0228102 A1
(43) Pub. Date: Jul. 29, 2021

(54) PULSE DEVICE AND METHOD TO INSTRUCT TREATMENT WITH PULSE DEVICE

(71) Applicant: Wellness Allied Inc, Natick, MA (US)

(72) Inventors: Weihui Li, Natick, MA (US); Tao ZHANG, Natick, MA (US)

(21) Appl. No.: 17/121,877

(22) Filed: Dec. 15, 2020

(51) Int. Cl. A61B 5/024; A61B 5/00; A61B 5/0255; G16H 10/60; G16H 50/30; G16H 40/63; G16H 50/20; G16H 70/20; G16H 20/40; G16H 20/30; G16H 20/90; G01F 1/34

(52) U.S. Cl. CPC A61B 5/02444; A61B 2562/0261; A61B 5/681; A61B 5/0255; A61B 5/742; A61B 5/6843; A61B 5/4854; G16H 10/60; G16H 50/30; G16H 40/63; G16H 50/20; G16H 70/20; G16H 20/40; G16H 20/30; G16H 20/90; G01F 1/34; A61B 5/02438

(57) ABSTRACT

A pulse device includes: force sensor arrays, force sensor carrier whose positions are manually adjustable to apply variable force on human body, a fixation structure to fix the force sensor carrier to human body, a control system controlling collection and transmission of data, a memory system, and a display system. A method including: measuring pulse waveforms; calculating characteristics of measured pulse waves; recommending one or more treatment methods to test on a human body for a few seconds to several minutes, and tracking the characteristics of the pulse wave during or after treatments to test their effectiveness; recommending one or more effective treatments for longer duration after finishing effectiveness tests; instructing the user to end treatments. The pulse device is convenient to use in clinic. It can also be developed into a wearable device to measure users' pulse in real time, and instructs users to perform self-care through software applications.

### TECHNICAL FIELD

The present invention generally relates to a pulse device and a method to instruct treatment based on the pulse wave characteristics measured by the pulse device.

### BACKGROUND

Chinese medicine has a history of more than two thousand years. Pulse diagnosis plays an important role in Chinese medicine. However, because it takes many years to train a TCM physician with the ability of pulse diagnosis, the pulse diagnosis in TCM treatment is not best used. With the advancement of science and technology, especially in the fields of sensors to measure forces and the microprocessors to process data, it has become possible for real-time pulse measurement, analysis and instruct the treatment of diseases.

There are many records about pulse diagnosis in Chinese medical literature. One of the most important sentences about pulse diagnosis is mentioned in a classic Chinese Medicine book called “Huang Di Nei Jing” that says “no matter what the patient's disease is, the treatment goal is to make the pulses at different positions balanced”.

In the past decades, a variety of pulse devices have been designed. CN102018501A mainly designs a visual positioning window for a pulse device for doctors to manually and conveniently locate the pulse locations. The pulse device is large in size and poor in portability. CN102258367A, CN1565378A, CN102579013B, CN105662368B, CN106264491A, CN101049247A, CN201624647U designed a group of pulse device stations. When in use, a hand needs to reach into the station. The disadvantage is that they are not portable or wearable; the station and the hand easily generate relative movement: if the wrist moves slightly, motion noises dominate and signals get lost.

CN104305971A proposed a pulse diagnosis method to determine a patient's constitution and health condition. In the patent, no pulse device was designed.

CN105147261B, CN107007269A, CN107440694A designed a group of pulse devices that use airbags to apply force. The disadvantage is that the devices need to be inflated for each measurement, which are time-consuming and the sizes are big with air pumps.

CN105433915A designed a pulse device with motors to apply force. The disadvantage is that the instrument is large in size and cannot be used quickly to measure pulse in the treatment rooms.

CN106419859A designed a wearable pulse bracelets and gloves, which can be used to measure pulse, body temperature, hemoglobin, etc., and can transfer the measured value through wireless transmission. However, there is no specific structure and method for applying force to the force sensors.

CN206473309U has designed a fully automatic portable pulse device that uses small motors to apply force during measurement. This type of design is expensive to manufacture.

CN2255818Y is a wearable pulse device that has three force sensors and are fixed in the instrument with springs. The disadvantage of the instrument is that, the force sensors are single force sensors instead of sensor array and cannot monitor the pulse waveform in spatial domain. Also, during the measurement, the force sensors are pressed on the radial artery all the time and might affect blood circulation.

The above mentioned inventions have different disadvantages: poor portability, heavy, not wearable, low accuracy for the force sensor, expensive. Also, none of them have ever proposed methods for treatment effectiveness testing facilitated by pulse devices.

### SUMMARY

Based on the past pulse devices, modern science and technologies, this patent designs a pulse device that is convenient to use in the treatment room or in daily life as a wearable device. The patent also presents a method to instruct treatments based on Chinese medicine classic literature and measured pulse wave characteristics.

According to one aspect of the present invention, a pulse device is provided, including a force sensor array for measuring pulse waves of peripheral arteries; a force sensor carrier, on which the force sensors attaches, and whose position is manually adjustable to apply variable force on human body; a fixation structure that fixes the force sensor carrier on human body; a display system to display measured pulse waves, pulse-derived values, working status and/or charging status of the pulse device; a memory system to store pulse waves and/or pulse-derived values; a control unit to control pulse measurement, data transmission, data storage, battery charging, and/or the display.

Optionally, the pulse device's control unit is further configured to execute executable instructions stored in the memory system to perform following steps: obtaining pulse waves measured by the force sensor array ; calculating characteristics of the pulse waves in spatial, time and/or frequency domain; recommending one or more treatment methods on one or more parts of a human body for a few seconds to several minutes, and track characteristics of the pulse waves during or after treatments to test treatment effectiveness; recommending one or more effective treatments on one or more parts of a human body for longer duration after finishing all treatment effectiveness testing.

Optionally, the pulse device's control unit is further configured to execute executable instructions stored in the memory system to perform the following steps: obtaining pulse waves measured by the force sensor array; calculating the characteristics of the pulse wave in spatial, time and/or frequency domain; tracking the pulse wave characteristics intermittently or continuously when users perform treatments on one or more parts of a human body at their choices; informing the users whether the treatments are effective.

Optionally, the pulse device's force sensor is force sensitive resistors, capacitive force sensor, piezoelectric force sensor or strain gauge sensor.

Optionally, the treatment methods include acupuncture, massage, thermal therapy, magnetic therapy, electrical stimulation, laser therapy, and ultrasound therapy that are performed manually or through machines.

Optionally, the characteristics of the pulse wave include one or more of the following: differences between peaks and troughs, slopes of the pulse wave, widths, lengths and areas of the pulse wave in spatial domain, pressures of troughs, pulse wave speed, pulse wave spectrum distribution.

Optionally, the pulse device instructs users to end treatments according to one of following preset conditions: timing the treatments, and ending the treatments after a preset time durations; tracking pulse wave characteristics, and ending the treatments when the pulse wave characteristics reach a preset optimal state.

Optionally, the pulse device's fixation structure to fix the pulse device on human body part is in a form of a clip, and the clip has two clipping pieces.

Optionally, the pulse device's fixation structure to fix the pulse device on human body is in a form of a bracelet or a watch.

Optionally, the pulse device's force sensor carrier comprises a button on which the force sensor attaches, a hollow screw in which the button stays, a screw nut on the fixation structure with which the hollow screw engages; or the force sensor carrier comprises two or more independent sets of above-mentioned button, hollow screw, and screw nut, with a force sensor array attaches to each button; or the force sensor carrier is in the form of an elongated button, on which one or more force sensor arrays are fixed.

Optionally, the elongated button has a segment of indented neck, around the neck there is a stopper that is a part of the fixation structure, and a button spring is sheathed around the neck above the stopper to bounce the button away from the human body when the button is disengaged from the fixation structure until the button end is stopped by the stopper to prevent the button being pushed out of the device completely.

Optionally, the fixation structure is built with movable blocks and block springs that press the movable blocks against the button at rest state; there are sawtooth on contact surfaces of the movable blocks and the button to engage each other; the movable blocks have external force application points; when a force is applied to the external force application points of the movable blocks, the movable blocks are disengaged from the button and the button is pushed upwards by the button spring.

Optionally, the elongated button is connected with a pole screw and the pole screw engages with a compatible screw nut on the fixation structure; rotating the pole screw generates a linear motion and drives the elongated button up or down.

Optionally, the hollow screw and the button positions are adjusted in one of the following ways: with the buttons at higher positions, adjust the hollow screws' positions relative to the screw nuts until the buttons just touch the skin, then press all the buttons down; with the buttons at lower positions, adjust the hollow screws' positions relative to the screw nut until measured pulse wave having largest values.

Optionally, when a treatment method is tested on a body part, and the pulse characteristics become more balanced between different positions of Cun, Guan, Chi, the treatment method is judged to be an effective method and the body part is an effective site to be treated.

Optionally, when a treatment method is tested on a body part, and the pulse characteristics match a preset optimal pulse waveform characteristics, the treatment method is judged to be an effective method and the body part is an effective site to be treated.

Optionally, the fixation structure is a clip to clamp on the human wrist, and the clip includes an upper piece having the force sensor carrier on it and a lower piece in a shape of a wrist cushion to extend the wrist, so that the radial artery protrudes out more and the pulse wave is measured with better signals.

According to another embodiment of the present invention, a method of instructing treatments based on a pulse system is provided, including: measuring pulse waveforms by a pulse device; calculating characteristics of the measured pulse waves in spatial, time and/or frequency domain, wherein the pulse wave characteristics include one or more of followings: differences between peaks and troughs, slopes of the pulse waves, widths, lengths and areas of the pulse wave in spatial domain, pressures of the troughs, pulse wave speeds, pulse wave spectrum distribution; recommending one or more treatment methods to test on one or more parts of a human body for a few seconds to several minutes, and tracking the characteristics of the pulse wave during or after treatments to test their effectiveness, wherein treatment methods include acupuncture, massage, thermal therapy, magnetic therapy, electrical stimulation, laser therapy, and ultrasound therapy; recommending one or more effective treatments on one or more parts of a human body for longer duration after finishing treatment effectiveness tests, wherein, the effectiveness is defined as one of following two criteria: the pulse characteristics become more balanced between different positions of Cun, Guan, Chi; the pulse characteristics match a preset optimal pulse waveform characteristics.

Optionally, the pulse device to facilitate the method of instructing treatments is any pulse device that measures the pulses from Cun, Guan, Chi on a radial artery and that is available on market, including the pulse device invented in present patent.

Optionally, the method of instructing treatments with a pulse device further comprise: instructing the user to end treatment(s) according to one of two preset conditions: timing the treatment, and ending the treatments after a preset time duration; tracking pulse wave characteristics, and ending the treatments when the pulse wave characteristics reach an preset optimal state.

According to the pulse device with the positions of the force sensor array adjustable, the device allows to apply force conveniently to start the pulse measurement. The user can also conveniently remove the force when the measurement is paused, so that the device will not affect the blood circulation. According to the method to instruct treatment based on the pulse device, the device calculates the pulse wave characteristics in the spatial, time and frequency domains and recommends for treatment methods and treatment location for efficacy testing. Eventually, the device can recommend efficient method to treat on efficient body location to achieve best treatment results.

### BRIEF DESCRIPTION OF THE DRAWINGS

FIG. 1A shows a schematic diagram of a pulse device that can be clamped on the wrist according to an embodiment of the present invention. FIG. 1B and 1C show in more detail the parts of 110, 120, 130 and 140 in FIG. 1A. FIG. 1B is a schematic diagram with the button not being pressed down, and the button end 140 with force sensor attached being not in contact with the wrist or slightly touching the wrist, and FIG. 1C is a schematic diagram after the button is pressed down, and there is a stronger contact force between the force sensor and the wrist at this time. FIG. 1D is a real press button product available in the market that is similar to the button in the present invention.

FIG. 2A shows another schematic diagram of a pulse device according to an embodiment of the present invention. The feature of this clip is that the clip piece without the force sensor can be made into different shapes, like a wrist cushion 210, so that the hand is extended and the radial artery protrudes out more. The pulse wave can be measured with better signals. In traditional Chinese medicine, a wrist cushion is generally placed under the wrist (as shown in FIG. 2B).

FIGS. 3A and 3B, from different angles, show another schematic diagram of a pulse device that can be clamped on the wrist according to an embodiment of the present invention. FIGS. 3C and 3D are longitudinal cross-sectional views along the dashed line 395 in FIG. 3A, which show in more detail the structural diagrams of 310, 320, 330, 340 in FIGS. 3A and 3B. FIG. 3C shows a state where the button 310 are engaged with the movable block 324 and the button end 340 with force sensor array attached is pressed down on human skin 347. FIG. 3D shows a state where the button 310 and the block 324 are disengaged, and the button end 340 is pushed away from the wrist by the button spring 345.

FIGS. 4A and 4D show two other types of pulse devices that can be clamped on the wrist according to embodiments of the present invention. 4B and 4E are longitudinal cross-sectional views along the dashed line 445 in FIGS. 4A and 4D, showing in more detail the structural diagrams of 410, 420, 430, 440, 425, 435 and 450 in FIGS. 4A and 4D. FIG. 4C shows in an enlarged view of the structure in which the elongated button 410 and the pole screw 430 in FIG. 4B are linked through the pole screw end 485 and groove 475.

FIGS. 5A and 5B show schematic diagrams of a pulse device in the form of a watch according to an embodiment of the present invention. 510 has the same button mechanism as in FIGS. 1, and 550 has the same button structure as in FIG. 3.

FIGS. 6A and 6B show a schematic diagram of force sensor array(s) according to an embodiment of the present invention. The array of FIG. 6A is suitable for use on the pulse clip shown in FIG. 1, and the sensor array of FIG. 6B is suitable for use on the pulse clip shown in FIGS. 3 and 4.

FIG. 7 shows an example of a pulse waveform measured by a pulse device according to an embodiment of the present invention. 710 is the pulse at the Cun position in Chinese medicine, 720 is the pulse at the Guan position, and 730 is the pulse at the Chi position.

FIG. 8A shows an example of features or parameters calculated for the pulse wave according to an embodiment of the present invention, including pulse magnitude H, area A, length L, and width W. FIG. 8B is time series of a pulse measured by one force sensor of the pulse device according to an embodiment of the present invention. FIG. 8C is the frequency spectrum obtained from the Fourier transform of FIG. 8B. The upper line is the real part of the Fourier transform, and the lower line is the imaginary part of the Fourier transform.

FIG. 9 shows an overall flow chart of a method 900 for guiding treatment with an aid of a pulse device according to an embodiment of the present invention.

### DETAILED DESCRIPTION

The embodiments of the present invention will be described in detail below with reference to the drawings.

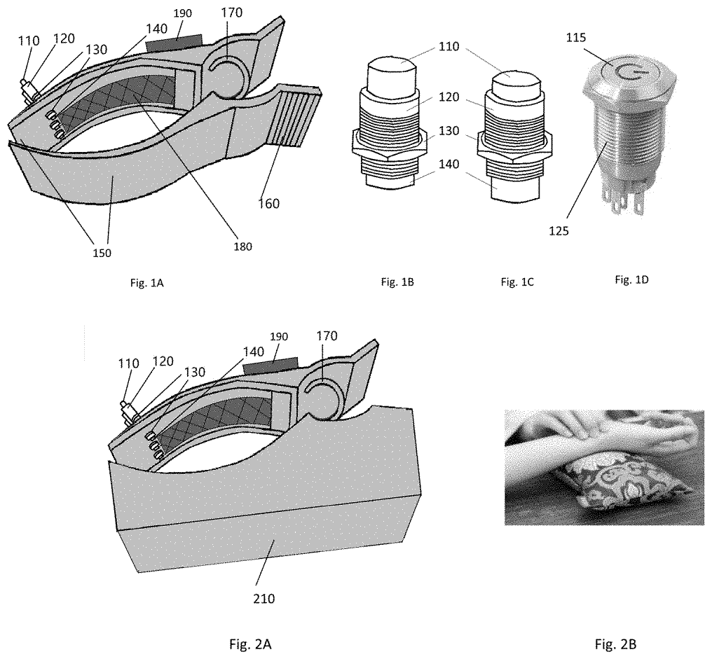

FIG. 1A shows a schematic diagram of a pulse device in a form of a clip that can be clamped on the wrist according to an embodiment of the present invention. The pulse device shown in FIG. 1A can be used to measure the pulse wave of a radial artery. A clip can be used for each hand so that the pulse waves of both hands can be measured at the same time. In traditional Chinese medicine, the radial artery proximal to the wrist is divided into Cun, Guan and Chi positions. The Cun, Guan, Chi of the left hand are empirically found to be related to the heart, liver, kidney-yin, and the Cun, Guan, Chi of the right hand are empirically found to be related to the lung, spleen, kidney-yang. Clamping on the left and right wrists with two pulse devices can simultaneously measure the pulse wave characteristics of the heart, liver, spleen, lung, kidney yin, and kidney yang. The clip of the pulse device can be designed into different sizes to match the patient's arm lengths and sizes.

In FIG. 1A, 110 is a button that can generate displacement, 120 is a hollow screw, 130 is a screw nut fixed on the clip, and 140 is a button end of the button 110 to with a force sensor array attached. 150 are the two clip pieces, 160 is handle to open the clip, 170 is the mechanical structure used to open or close the clip. 180 are the wires connecting the force sensor array and the control circuit board 190. Before use, select the appropriate size of the pulse device according to the patient's arm length and size, and put the button and hollow screw in higher positions without pressing or screwing down. When in use, open the clip by pressing the handles 160, align the buttons 140 with the patient's Cun, Guan, Chi positions, adjust the positions of the hollow screws 120 and buttons 110 and turn on the pulse device. The force sensor arrays attaching to the button end 140 of the button 110 start to measure pulse waves.

An example of adjusting the hollow screw 120 and the button 110 is to adjust the hollow screw first so that the force sensor arrays just touches the skin, and the static contact force is around 0 Newton. Then press down all three buttons (FIG. 1B shows the higher state and FIG. 1C shows the lower state) which will generate a static force greater than 0 Newton on the radial artery as well as a dynamic pulse pressure. The hollow screw 120 and the button 110 can also be adjusted according to the waveform of the pulse wave: press down the buttons 110, and then adjust the hollow screw to maximize the collected pulse wave.

It should be noted that in the example shown in FIG. 1A, three force sensor arrays are arranged on the three buttons 110. This is designed to measure the pulse pressure on Cun, Guan, Chi positions. This is only an example and the number of buttons can be set differently with force sensor arrays attached on each of these buttons.

FIG. 1B and 1C show in more detail the parts of 110, 120, 130 and 140 in FIG. 1A. FIG. 1B is a schematic diagram of the button not being pressed down, and the button end 140 is not in contact with the wrist or slightly touching. FIG. 1C is a schematic diagram after the button is pressed down, and there is a stronger contact force between the force sensor and the wrist at this time. FIG. 1D is a real press button product available in the market with button 115 and hollow screw 125 that are similar to the button and hollow screw used in the present invention.

FIG. 2A shows another schematic diagram of a pulse device according to an embodiment of the present invention. The feature of this clip is that the clip piece without the force sensor can be made into different shapes, like a wrist cushion 210, so that the radial artery protrudes out more and the pulse wave is measured with better signals. In traditional Chinese medicine, a wrist cushion is generally placed under the wrist (as shown in FIG. 2B).

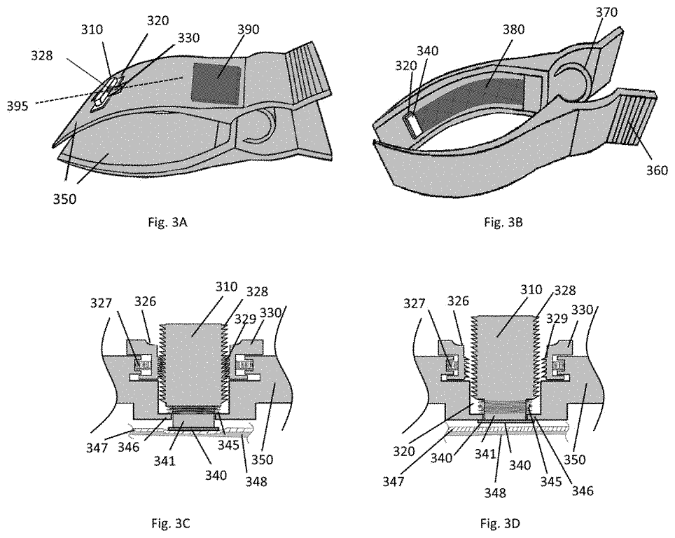

FIGS. 3A and 3B show another pulse device that can be clamped on the wrist when in use according to an embodiment of the present invention. The device can also be worn on each of the two wrists to collect the pulse waves of both hands. It can also be designed in various sizes according to the arm length and sizes. The pulse device shown in FIG. 3A is different from that shown in FIG. 1A in that the three independent buttons in FIG. 1A are replaced by an elongated button in FIG. 3A.

In FIG. 3A, 310 is an elongated button that can produce displacement; 320 is an opening on the clip to fit the button 310; 330 are movable blocks to engage or disengage the button 310 through sawtooth structures 328, 329 (shown in FIGS. 3C and 3D) on the contact surface of the button and the movable blocks; 340 is the button end with force sensor array attached; 350 are two clip pieces; 360 is the handle to open the clip; 370 is the mechanical structure to open or close the clip when pressing on the handle 360. 380 are the wires connecting the force sensor array and the control circuit 390.

FIGS. 3C and 3D are longitudinal cross-sectional views along the dashed line 395 in FIG. 3A, showing in more detail the structural diagrams of 310, 320, 330, and 340 in FIGS. 3A and 3B. There is an indented neck 341 on the button 310. Around the indented neck there are a stopper 346 that is part of the clip and a button spring 345. 328 is the sawtooth on both sides of the button 310, which can be engaged with the sawtooth 329 on the movable block 324 to lock the button at different depth. 326 is the force application point of the block 324, which is used to disengage the movable block 324 and the button 310. 327 are block springs that push the block 324 towards 310 at its rest state. When the blocks are pushed outwards by applying force on 326, the block springs 327 are compressed that disengages the sawtooth 329 and 328. At this moment, the button spring 345 pushes 310 upwards to releases the pressure on the wrist. The button end 340 is wider than the neck 341 and the opening of the stopper 346, so that the stopper 346 can block the button end 340 to prevent 310 from being completely ejected by the button spring 345. FIG. 3C shows a state where the button 310 is engaged with the block 324, the button end 340 is pressed against the human skin 347 and the artery 348 is slightly squeezed. FIG. 3D shows a state when the button 310 and the block 324 are disengaged, and 340 is moved away from the human skins.

An example of using the pulse device according to an embodiment of the present invention is given below. Before use, select the appropriate size of the pulse device, at this time the button is not pressed down. When in use, open the clip, align the button on the positions of Cun, Guan, Chi of the wrist, turn on the pulse device and start collecting data. Then press down the button slowly, and the system will remind the user to stop pressing the button according to the magnitude of the static force and the dynamic force. At this time, pulse waves are measured and acupuncture or massage treatments can be performed according to the pulse wave.

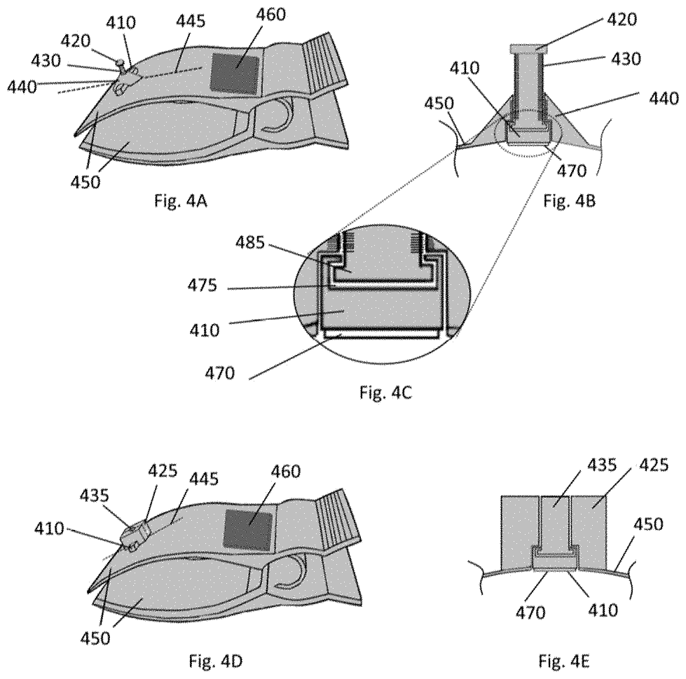

FIGS. 4A and D show two other types of pulse devices that are clamped on the wrist. 450 are the clip pieces, 460 is the control circuit board, and 410 is an elongated button. FIGS. 4B and 4E are longitudinal cross-sectional views along the dashed line 445 in FIGS. 4A and 4D, showing in more detail the structural diagrams of 410, 420, 430, 440, 425, 435 and 450 in FIGS. 4A and 4D. 420 is a screw head and 430 is pole screw, 440 is a screw nut on the clip piece 450 that is engaged with the pole screw 430, 470 is a force sensor array attaching to one end of the elongated button. On the other end of the button 410 is a groove structure 475, shown in an enlarged view in FIG. 4C. The button 410 and the pole screw 430 are linked through the screw end 485 and groove 475, which also allows the pole screw to rotate. When the pole screw 430 is rotated, the pole screw can move the elongated button 410 up or down.

In FIGS. 4D and 4E, 425 is a linear motor, and 435 is a rod inside the linear motor that can move linearly. The end of the rod linked with the button in a same way as shown in FIG. 4C. The rod can be driven by the motor to move the button up and down. According to an embodiment of the present invention, the pulse device driven by the motor to move the button can be used to guide the treatment.

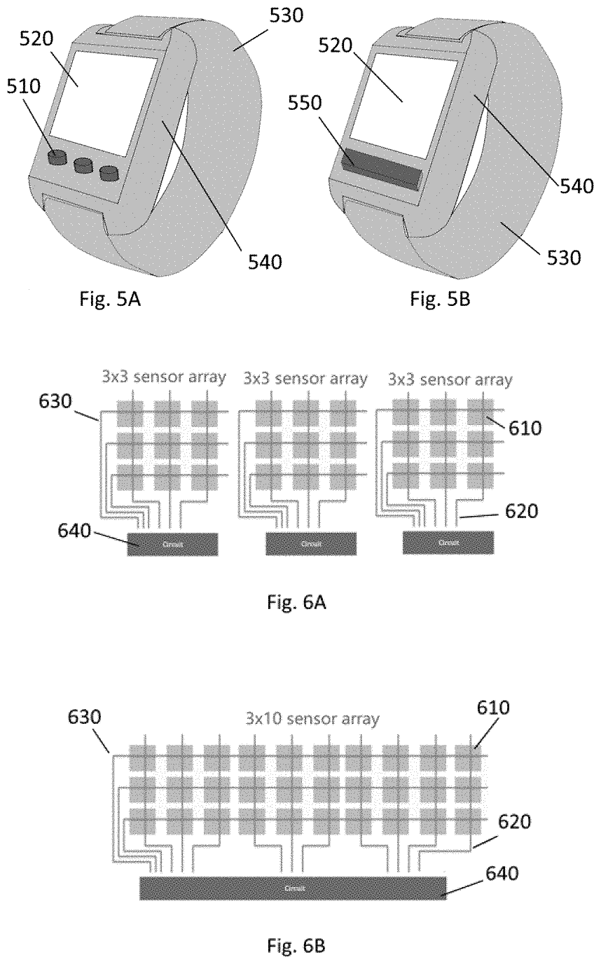

FIGS. 5A and 5B show schematic diagrams of a pulse device in the form of a wristwatch according to an embodiment of the present invention. 510 has the same button structure as in FIG. 1, including a hollow screw, a screw nut, a button, and a sensor array. 550 has the same button structure as in FIGS. 3 and 4. 520 are the display screens, 530 are the straps, and 540 are the covers. When in use, put the watch on the wrist, and assign the button 510 or button 550 with the Cun, Guan, Chi positions of the radial artery. As of a note, in FIGS. 5A and 5B, the buttons are on the wristwatch face. But the buttons can also be integrated on wristwatch's straps.

6A and 6B show schematic diagrams of force sensor arrays according to an embodiment of the present invention. The array shown in FIG. 6A is suitable for use on the pulse device shown in FIG. 1A, and the sensor array of FIG. 6B is suitable for use on the pulse device shown in FIGS. 3 and 4. 610 is individual force sensor unit, 620 and 630 are the column and row selection lines. By selecting one row and one column, the pressure value on one force sensor can be measured. FIG. 6A shows 3 arrays, each with 3 rows and 3 columns, and FIG. 6B shows a 3x10 sensor array. The above-mentioned sensor array is only an example, and other number of sensor arrays can be used in the specific pulse devices.

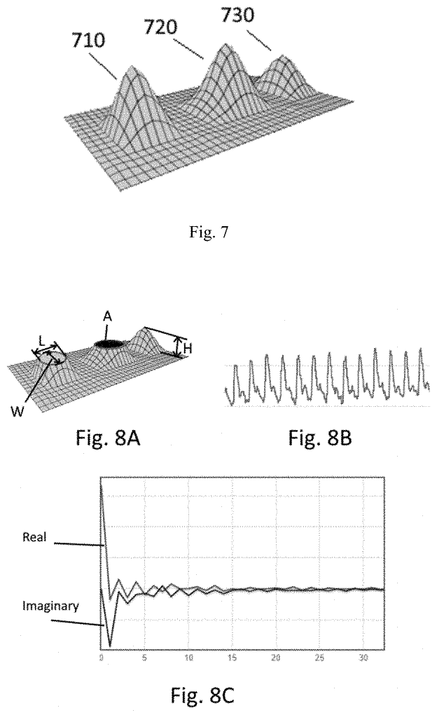

FIG. 7 shows an example of a pulse waveform based on the signals measured from the pulse device shown in FIG. 1A. The pulse waveform is plotted with interpolation and graphic processing. 710 is the Cun pulse in Chinese medicine, 720 is Guan pulse, and 730 is Chi pulse.

FIG. 8A shows an example of characteristics or parameters calculated for the pulse waveform according to an embodiment of the present invention, including pulse magnitude H, area A, length L, and width W, wherein the area, length and width are measured when the pulse has a value half of the pulse magnitude. In other specific implementation process, other values can be chosen to calculate the area, length, and width of the pulse waveform. FIG. 8B is a time series of pulse waves measured by one force sensor in the pulse sensor array according to an embodiment of the present invention. FIG. 8C is the pulse spectral value obtained by the Fourier transform of FIG. 8B, with the upper line being the real part of the Fourier transform, and the lower line being the imaginary part of the Fourier transform. However, the spectral analysis method is not limited to Fourier Transform. Other methods, such as wavelet analysis, Hilbert-Huang transform can also be used. In addition, for each moment, the spatial distribution of the pulse wave collected from each force sensor can be analyzed in the frequency domain to obtain the spatial spectrum value.

One or more of the above parameters can be used to recommend specific treatments on specific parts of the human body and test the effectiveness of these treatment methods and treatment parts according to changes in the characteristic values of pulse waves. Treatment methods may include: acupuncture, massage, thermal therapy, magnetic therapy, electrical stimulation, laser therapy, or ultrasound therapy.

Exemplarily, a test process according to an embodiment of the present invention includes: recommending one or more body parts to sequentially perform acupuncture, massage, thermal therapy, magnetic therapy, electrical stimulation, laser, or ultrasound therapy for a few seconds to a few minute; the system instructing to stop the therapy on the current body part and to test the next body part; evaluating the effectiveness of the therapy on body parts by analyzing the characteristic parameters of the pulse wave. After testing all recommended treatment methods and treatment parts, the system recommends one or more effective treatment methods on one or more effective body parts.

Judging whether a treatment method is effective in different parts includes: measuring and calculating the pulse wave characteristics from different parts, for example, Cun, Guan and Chi positions. If the pulse wave characteristics of different parts become more balanced as the treatment progresses, then the method is judged to be an effective method, this body part is an effective treatment location; when testing a treatment method on a body part, if the pulse wave characteristics match a preset normal waveform characteristics, then this method is judged to be an effective method and this body part is the effective part.

One or more of the above parameters can also be continuously displayed to instruct users to determine specific treatment methods for specific parts of the body based on their own experience, and according to the pulse wave characteristics in spatial, time and/or frequency domain.

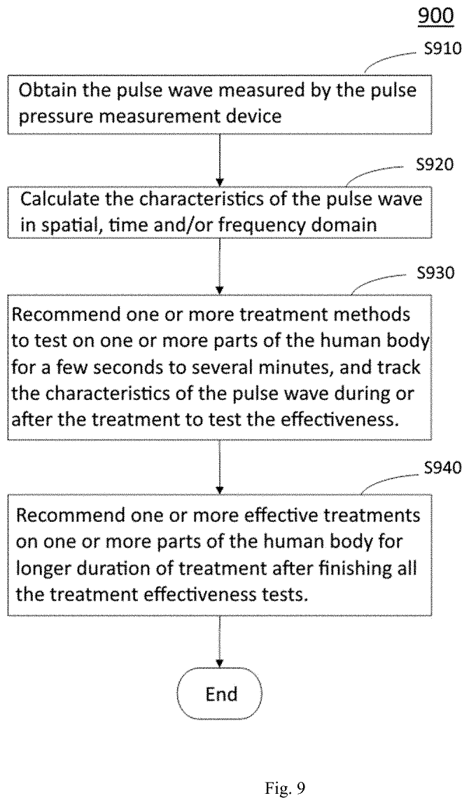

FIG. 9 shows an overall flowchart of a method 900 for guiding treatment in combination with a pulse device according to an embodiment of the present invention. The treatment methods may include acupuncture, massage, thermal therapy, magnetic therapy, electrical stimulation, laser therapy, and ultrasound therapy that are performed manually or through machines.

As shown in FIG. 9, in step S910, the pulse waveform is measured by the pulse device.

In step S920, the characteristics of the measured pulse wave in spatial, time and/or frequency domain are calculated. The characteristics include one or more of the following: the difference between the peak and the trough, the slope of the pulse wave, the width, length and area of the pulse wave, the pressure of the trough, the propagation velocity, and the frequency spectrum.

In step S930, the distribution of each feature value is calculated, and then one or more treatment methods are recommended to test on one or more parts of the human body for a few seconds to several minutes, and track the characteristics of the pulse wave during or after the treatment to test the effectiveness.

In step S940, after testing all the recommended treatment methods and treatment parts, the system recommends one or more effective treatment methods on one or more effective body parts for longer duration after finishing all the treatment effectiveness tests.

For example, when testing a treatment method on a body part, measure and calculate the pulse wave characteristics. If it is found that the pulse wave characteristics from positions of Cun, Guan, Chi become more balanced as the treatment progresses, then this method is judged to be effective method, this body part is the effective treatment part.

For another example, when a treatment method is tested on one or more body parts and the pulse wave characteristics match the preset normal pulse waveform characteristics, then this method is judged to be an effective method and the body part is an effective treatment part.

In an example, the method for guiding treatment further includes: instructing the user to end treatment according to a set conditions, and the set condition is one of the followings: timing the treatment, and ending the treatments after a preset time duration; tracking pulse wave characteristics, and ending the treatments when the pulse wave characteristics reach an preset optimal state.

The method of guiding treatments can be executed by a pulse device, or other computing devices capable of communicating with the pulse device, such as a smart phone.

According to the pulse device for diagnosis and the method for guiding treatments according to the embodiment of the present invention, the position of the force sensor of the pulse devices can be adjusted, to facilitate more flexible and accurate measurement of human pulse, calculate the characteristics of the pulse waves in spatial, time and frequency domains, and give recommendations for effective treatment methods and treatment sites by tracking the pulse wave changes during the test.

The embodiments of the present invention have been described above. The above description is exemplary, not exhaustive, and is not limited to the disclosed embodiments. Many modifications and variations will be apparent to those of ordinary skill in the art without departing from the scope and spirit of the illustrated embodiments. Therefore, the protection scope of the present invention should be subject to the protection scope of the claims.

1. A pulse device, including:
a force sensor array for measuring pulse waves of peripheral arteries;
a force sensor carrier, on which the force sensor array attaches, and whose position is manually adjustable to apply variable force on human body;
a fixation structure that fixes the force sensor carrier on human body;
a display system to display measured pulse waves, pulse-derived values, and/or working status of the pulse device;
a memory system to store pulse waves and/or pulse-derived values;
a control unit to control pulse measurement, data transmission, data storage, and/or the display.

2. The pulse device according to claim 1, wherein the control unit is further configured to execute executable instructions stored in the memory system to perform following steps:
obtaining pulse waves measured by the force sensor array ;
calculating characteristics of the pulse waves in spatial, time and/or frequency domain;
recommending one or more treatment methods on one or more parts of a human body for a few seconds to several minutes, and track characteristics of the pulse waves during or after treatments to test treatment effectiveness;
recommending one or more effective treatments on one or more parts of a human body for longer duration after finishing all treatment effectiveness testings.

3. The pulse device according to claim 1, wherein the control unit is further configured to execute executable instructions stored in the memory system to perform the following steps:
obtaining pulse waves measured by the force sensor array;
calculating the characteristics of the pulse wave in spatial, time and/or frequency domain;
tracking the pulse wave characteristics intermittently or continuously when users perform treatments on one or more parts of a human body at their choices;
informing the users whether the treatments are effective.

4. The pulse device according to claim 1, wherein the force sensor is force sensitive resistors, capacitive force sensor, piezoelectric force sensor or strain gauge sensor.

5. The pulse device according to claim 2, wherein the treatment methods include acupuncture, massage, thermal therapy, magnetic therapy, electrical stimulation, laser therapy, and ultrasound therapy.

6. The pulse device according to claim 2, wherein the characteristics include one or more of the following: differences between peaks and troughs, slopes of the pulse wave, widths, lengths and areas of the pulse wave in spatial domain, pressures of troughs, pulse wave speed, pulse wave spectrum distribution.

7. The pulse device according to claim 2, further comprising: instructing users to end treatments according to one of following preset conditions: timing the treatments, and ending the treatments after a preset time duration; tracking pulse wave characteristics, and ending the treatments when the pulse wave characteristics reach a preset optimal state.

8. The pulse device according to claim 1, wherein the fixation structure to fix the pulse device on human body is in a form of a clip, and the clip has two clipping pieces.

9. The pulse device according to claim 1, wherein the fixation structure to fix the pulse device on human body is in a form of a bracelet or a watch.

10. The pulse device according to claim 1, wherein the force sensor carrier comprises a button on which the force sensor attaches, a hollow screw in which the button stays, a screw nut on the fixation structure with which the hollow screw engages; or
the force sensor carrier comprises two or more independent sets of above-mentioned button, hollow screw, and screw nut, with a force sensor array attaches to each button; or
the force sensor carrier is in the form of an elongated button, on which one or more force sensor arrays are fixed.

11. The pulse device according to claim 10, wherein the elongated button has a segment of indented neck, around the neck there is a stopper that is a part of the fixation structure, and a button spring is sheathed around the neck above the stopper to bounce the button away from the human body when the button is disengaged from the fixation structure until the button end is stopped by the stopper to prevent the button being pushed out of the device completely.

12. The pulse device according to claim 11, wherein the fixation structure is built with movable blocks and block springs that press the movable blocks against the button at rest state;
there are sawtooth on contact surfaces of the movable blocks and the button to engage each other; the movable blocks have external force application points; when a force is applied to the external force application points of the movable blocks, the movable blocks are disengaged from the button and the button is pushed upwards by the button spring.

13. The pulse device according to claim 10, wherein the elongated button is connected with a pole screw and the pole screw engages with a compatible screw nut on the fixation structure; rotating the pole screw generates a linear motion and drives the elongated button up or down.

14. The pulse device according to claim 10, wherein the hollow screw and the button positions are adjusted in one of the following ways:
with the buttons at higher positions, adjust the hollow screws' positions relative to the screw nuts until the buttons just touch the skin, then press all the buttons down;
with the buttons at lower positions, adjust the hollow screws' positions relative to the screw nut until measured pulse wave having largest values.

15. The pulse device according to claim 2, wherein when a treatment method is tested on a body part, and the pulse characteristics become more balanced between different positions of Cun, Guan, Chi, the treatment method is judged to be an effective method and the body part is an effective site to be treated.

16. The pulse device according to claim 2, wherein when a treatment method is tested on a body part, and the pulse characteristics match a preset optimal pulse waveform characteristics, the treatment method is judged to be an effective method and the body part is an effective site to be treated.

17. The pulse device according to claim 8, wherein the clip is used to clamp on the human wrist, and includes an upper piece having the force sensor carrier on it and a lower piece in a shape of a wrist cushion to extend the wrist, so that the radial artery protrudes out more and the pulse wave is measured with better signals.

18. A method of instructing treatments with a pulse device, including:
measuring pulse waveforms by a pulse device;
calculating characteristics of the measured pulse waves in spatial, time and/or frequency domain, wherein the pulse wave characteristics include one or more of followings: differences between peaks and troughs, slopes of the pulse waves, widths, lengths and areas of the pulse wave in spatial domain, pressures of the troughs, pulse wave speeds, pulse wave spectrum distribution;
recommending one or more treatment methods to test on one or more parts of a human body for a few seconds to several minutes, and tracking the characteristics of the pulse wave during or after treatments to test their effectiveness, wherein treatment methods include acupuncture, massage, thermal therapy, magnetic therapy, electrical stimulation, laser therapy, and ultrasound therapy;
recommending one or more effective treatments on one or more parts of a human body for longer duration after finishing treatment effectiveness tests, wherein, the effectiveness is defined as one of following two criteria: the pulse characteristics become more balanced between different positions of Cun, Guan, Chi; the pulse characteristics match a preset optimal pulse waveform characteristics.

19. The method of instructing treatments with a pulse device according to claim 18, further comprising: instructing the user to end treatment(s) according to one of two preset conditions: timing the treatment, and ending the treatments after a preset time duration; tracking pulse wave characteristics, and ending the treatments when the pulse wave characteristics reach a preset optimal state.
